(19) United States
(12) Patent Application Publication
(10) Pub. No.: US 2007/0016171 A1
(43) Pub. Date: Jan. 18, 2007

(54) Implantable perfusion device

(76) Inventors: Jean-Marie Podvin, Beuvry La Foret (FR); Yves Lecoffre, Grenoble (FR)

Correspondence Address: LE-NHUNG MCLELAND, 9679-C MAIN STREET, FAIRFAX, VA, 22031-3766, US

(21) Appl. No.: 10/575,233

(22) PCT Filed: Sep. 30, 2004
(86) PCT No.: PCT/FR04/02478

§ 371 Date: Apr. 7, 2006

(30) Foreign Application Priority Data
Oct. 7, 2003 (FR) 0311705

(51) Int. Cl. A61K 9/22

(52) U.S. Cl. 604/891.1

(57) ABSTRACT

The present invention relates to an implantable device for continuously administering liquid medication to a patient at a very low rate. The device of the invention comprises: a transfer chamber enabling a supply to be fed with liquid; transfer means for transferring said liquid between the transfer chamber and said supply; and a supply enabling said liquid to be delivered, the supply including: an inlet for receiving said liquid; and at least one outlet for delivering said liquid to the patient.

The present invention relates to devices for perfusing liquid medications, and more particularly it relates to an implantable device for continuously administering medication to a patient at a very small dosage rate.

### BACKGROUND OF THE INVENTION

Certain chronic illnesses, such as diabetes, cancer, AIDS, or diseases of the blood, require perfusions to be performed on a continuous basis in order to administer medication to the patient during periods of time covering the range a few months to a few years.

Several known perfusion systems exist at present:
the stationary type, generally used in hospitals, where the medication flows under gravity, e.g. from plastics bags hung up above the patient's bed; such systems present the major disadvantage of requiring the patient to remain in bed, and consequently are severely limited in terms of duration of use;
of the ambulatory type, where the medication is administered by means of a motor-driven pump that the patient wears or carries, enabling the medication to be injected automatically in the patient's body; the drawbacks of this solution lie in the relatively great weight of the pump, and in the inconvenience caused by the tube connecting the pump to the site where the medication is injected, generally situated in the patient's abdomen.

As a result, a solution relying on a pump that can be implanted in the patient's body is becoming essential, because of the many advantages it provides:
high level of safety;
greater comfort for the patient, enabling the patient to lead a normal life;
the medical assistance required by the patient is reduced to a minimum.

The implantable perfusion devices known in the prior art comprise a perfusion pump having a supply of medication, said pump being placed under the patient's skin in a bag generally situated in the abdomen; the filler orifice of said supply is detected by palpating the patient's skin. Said supply, which is at negative pressure, is filled periodically with medication:
either by means of a needle connected to a syringe containing the liquid medication; under such circumstances, the needle passes through the patient's skin, then the filler orifice, and then opens out into the supply, with the liquid being delivered under pressure exerted on the syringe;
or else by means of a periodic refiller device, e.g. a capsule, containing the liquid to be perfused, which is secured in leaktight manner to the filler orifice, in which case the liquid is sucked into the supply by the negative pressure that exists therein.

Those prior implantable perfusion devices present several drawbacks:
the location under the patient's skin in the region of the abdomen can be inconvenient, particularly in the long term;
in order to make the operation of filling said supply of medicine as safe as possible, such devices present structures that are very complex, thereby leading to significant extra manufacturing cost;
the supply must be at negative pressure in order to ensure there is no leakage of the liquid that is to be perfused into the body of the patient; and
since filling said supply with medication is an operation that is very tricky, it requires special competence on the part of the medical personnel carrying it out.

### OBJECT AND SUMMARY OF THE INVENTION

The present invention seeks to remedy the above-mentioned drawbacks by creating an implantable device for perfusing liquid medication, comprising a supply of simple construction suitable for placing in a cavity of the body that is suitably selected to ensure comfort for the patient, the medication chamber of said supply being refilled periodically in a manner that is simple and safe.

According to the invention, the implantable device for administering a liquid, in particular a medication in man or animal comprises:
a transfer chamber enabling a supply to be fed with liquid;
transfer means for transferring said liquid between the transfer chamber and said supply; and
a supply enabling said liquid to be delivered, the supply including:
   an inlet for receiving said liquid; and
   at least one outlet for delivering said liquid to the patient.

The device of the invention is for delivering a basal physiological quantity of medication via the central vascular system, in particular for delivering insulin to insulin-dependent diabetic patients, on a continuous basis. The device comprises an implantable chamber of small dimensions which feeds a supply via a tube made safe by means of a check valve, which supply continuously delivers a constant quantity that is physiologically active for a given patient (where the quantity of active principals delivered is a function of the weight of the patient and of the dilution of said active principals in the injected solution). The supply is connected to a perfusion catheter via a very small diameter capillary of great length, thereby ensuring a large amount of head loss as the fluid passes. Said capillary is provided with control means enabling the flow to be stopped or started again, which means comprise a remotely-controlled valve and means for controlling said valve. Means for measuring the liquid flow rate are provided within said capillary.

### BRIEF DESCRIPTION OF THE DRAWINGS

The invention can be better understood on reading the following description made with reference to the accompanying drawings, in which:

FIG. 1 is a diagram of the transfer chamber and the means for transferring liquid to the supply;

FIG. 2 is a diagram in cross-section showing a pressurized supply in a particular embodiment of the invention; and

FIG. 3 is a diagram of an implantable perfusion device of the invention.

### MORE DETAILED DESCRIPTION

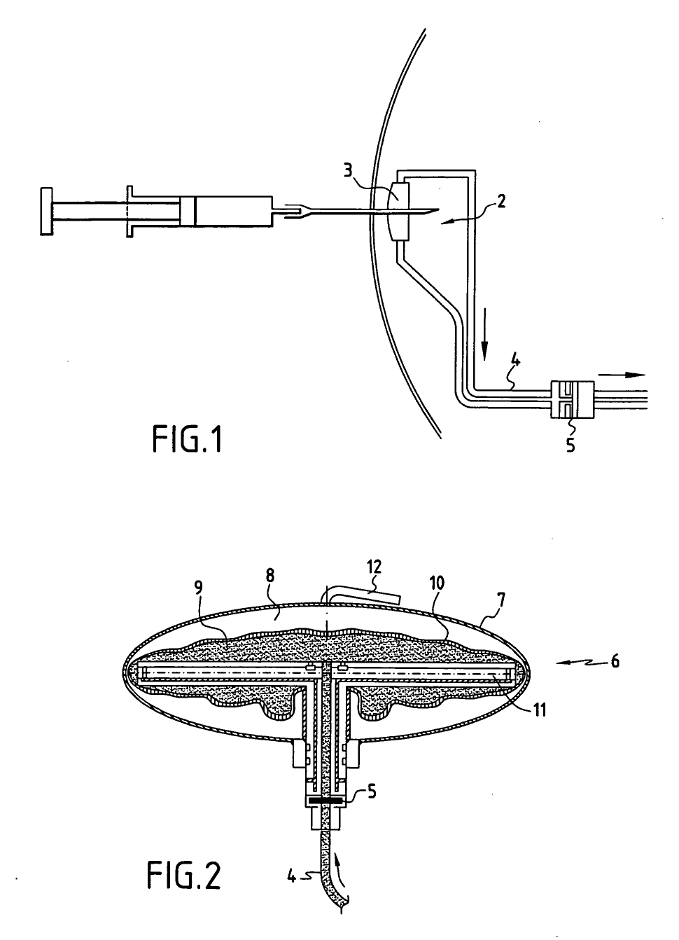

As shown in FIG. 1, the device 1 of the invention comprises a transfer chamber 2 implanted under the patient's skin. The filler orifice of the chamber 2 is provided with a septum 3 through which the needle of a syringe or any other suitable means can deliver a liquid medication to the patient. The means for transferring the liquid between the chamber 2 and the supply that enables the medication to be delivered to the patient comprise a tube 4 with a check valve 5 installed in the tube 4 so as to allow liquid to pass only towards said supply, in the direction shown.

FIG. 2 shows a pressurized supply enabling liquid to be delivered to the patient in a particular embodiment of the invention. The supply 6 comprises a rigid casing 7 comprising a chamber 8 containing a phase-change fluid, within which there is the medication chamber 9 defined by a flexible membrane (bag) 10. In a particular embodiment, a capillary 11 for delivering the medication to the patient is placed inside the medication chamber 9. Filler means 12 are provided to allow the supply 6 to be filled with phase-change fluid prior to being put into service.

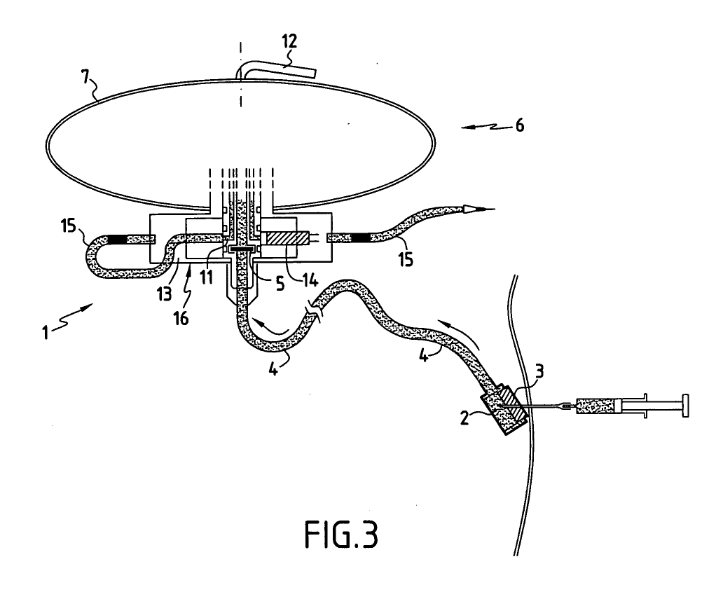

FIG. 3 shows the means for controlling the flow of liquid in the capillary 11, comprising in particular a valve 13 and means 14 for measuring the flow rate in the capillary 11. Liquid is fed via the tube 4 from the chamber 2. The liquid for perfusing is injected into the patient using a catheter 15, via its distal end. The catheter 15 is connected to the capillary 11 at the outlet from the supply 6.

The various elements involved in the structure of the device of the invention are described in detail below.

Transfer Chamber

The transfer chamber 2 is implanted surgically in the subcutaneous adipose tissues of the patient. The central portion of its leaktight housing is provided with a septum 3, and its side is provided with an outlet tube. The transfer chamber 2 is connected to the tube 4 that is made of a biocompatible material (e.g. radio-opaque polyurethane or silicone).

The proximal end of the tube 4 is connected to said outlet tube of the chamber 2. The connection between said outlet tube and the tube 4 is provided by a safety ring (not shown). The distal end of said tube 4 is connected to the supply 6 that is at constant pressure. At the junction between the tube 4 and the supply 6, there is the check valve 5.

The internal volume of the transfer chamber 2 is small compared with that of the medication chamber 9 of the supply 6; the purpose of the transfer chamber 2 is not to store the liquid that is to be perfused, but to transfer it to the supply 6 so as to enable said medication chamber 9 to be filled periodically. The liquid medication for perfusing is injected (after being filtered on biocompatible media and degassed), e.g. using a needle passing through the septum 3 and opening out into the transfer chamber 2, from which the liquid passes into the tube 4 and is subsequently stored in the medication chamber 9.

In order to avoid obstructing the tube 4, a sterile filter is placed at the outlet from the transfer chamber 2, e.g. where said tube 4 leaves the chamber 2.

It is possible to rinse the transfer chamber 2, e.g. by using two Huber needles simultaneously. The first needle is connected to a syringe that injects a cleaning liquid (e.g. a dilute solution of the liquid for administration to the patient). The second needle remains free and is used for removing the rinsing liquid. The pressure generated by the injecting needle must remain well below the pressure of the liquid contained in the supply 6.

Supply Enabling Liquid to be Delivered to the Patient

The supply 6 is implanted in the patient's body, at a location that is suitably chosen for patient comfort. Said supply may be placed at a distance lying in the range 0.5 centimeters (cm) to 40 cm from the transfer chamber 2.

The supply 6 comprises a rigid casing 7 made of a biocompatible material such as titanium, that is leakproof against the liquid for perfusing and against body fluids.

The casing corresponds to an enclosure having a diameter of 30 millimeters (mm) to 60 mm, a thickness of 10 mm to 20 mm, and a total weight (including the weight of the liquid for perfusing and of the capillary) of less than 60 grams (g).

The supply 6 comprises a chamber 9 containing the liquid for perfusing that is connected to a capillary 11.

The supply 6 can deliver said liquid to the patient even while being at negative pressure, in which case it is provided with means that ensure that the liquid contained in the chamber 9 flows at a constant rate along the capillary 11.

In a particular embodiment of the invention, the supply 6 comprises means for keeping the liquid contained in the chamber 9 at a constant pressure using techniques that are known to the person skilled in the art: a mechanical spring, compressed gas, evacuation of a liquid at constant temperature.

By being pressurized, the liquid can be caused to flow along the capillary 11. In principle, only a small pressure is required to obtain this flow once the capillary 11 has been completely filled with liquid. Pressure at the injection point can vary as a function of the position of the patient, and as a function of the possible development of a blood clot around the injection catheter. A pressure lying in the range a few bars to a few tens of bars is sufficient to compensate for these pressure differences.

In a preferred embodiment, the supply 6 is pressurized by making use of a phase-change fluid. Under such circumstances, the rigid casing 7 is leaktight against said phase-change fluid.

Phase-Change Fluid Chamber

The chamber containing the phase-change fluid chamber 8 contains a liquid-vapor mixture of a phase-change fluid whose pressure depends only on the practically constant temperature of the human body, i.e. about 37±1° C. The liquid for perfusion is subjected to this pressure through a flexible and leaktight membrane 10; the pressure of the liquid for injection thus itself becomes constant and equal to that of the liquid-vapor mixture. This propellant may be constituted by any substance having a liquid-vapor phase change with vapor pressure at 37° C. lying in the range a few bars to a few tens of bars. In an embodiment, the propellant gas is isobutane which has vapor pressure at 37° C. equal to 4.93 bars absolute. In another embodiment, the propellant gas may be propane, having vapor pressure at 37° C. of 12.75 bars.

When the supply 6 is pressurized by means of a phase-change fluid, the liquid in the transfer chamber 2 needs to be injected sufficiently slowly to ensure that the fluid contained in the constant pressure supply 6 has time to change phase. Even under these conditions, the time required to fill the medication chamber 9 via the transfer chamber 2 remains less than 10 minutes (min).

Medication Chamber

The medication chamber 9 is of limited capacity, e.g. 10 millimeters (mL) which corresponds to utilization between refills of two to four weeks for a delivery rate lying in the range 1 microliter per hour (μL/h) to 50 μL/h. The volume to be injected monthly is preferably 7 mL, which corresponds to a flow of about 10 μL/h.

In a preferred embodiment, the flexible bag 9 containing the liquid medication for injection and the coiled capillary 11 is placed inside the chamber 8 containing the phase-change fluid.

This configuration presents numerous advantages:
there is no contact between the liquid medication and the outer casing 7 of the device, thereby reducing any risk of the device being punctured;
there are no large forces to be applied locally to the membrane 10;
the pressure of the medication is indeed equal to the pressure of the phase-change fluid, since the bag 10 is highly deformable;
the casing 7 is filled with phase-change fluid after it has been closed;
the liquid medication is inserted after the casing 7 has been closed;
the membrane 10 prevents any foreign body being introduced that might have formed during welding or during assembly of the rigid casing 7; and
the contact area between the phase-change fluid and the tissue surrounding the supply 6 is maximized, thereby optimizing the transfer of heat energy between said tissues and the phase-change fluid, which is important, in particular while filling said supply with a liquid that is lower than that of the body, being at a temperature lying in the range about 4° C. to 25° C. As a result, the variations in the pressure of the phase-change fluid during filling remain negligible and do not lead to significant variations in the flow rate of the fluid for perfusing.
   System for Controlling Flow Rate by Implementing Controlled Head Loss

Flow rate is controlled by means of the capillary tube by implementing a supply 6 that is pressurized, containing the liquid for injection (as described above) in the chamber 9 and a coiled capillary tube 11 along which the liquid for injection travels flowing under lamellar conditions.

The characteristics of the capillary 11, are calculated on the basis of various parameters:
the magnitude of the injection flow rate, e.g. close to 2.7×10⁻¹² cubic meters per second (m³/s) for 100 unit insulin (corresponding to a monthly flow of about 7 mL);
the in-body operating temperature, which is close to 37° C.;
the dynamic viscosity of the liquid to be injected; it corresponds to 0.695×10⁻³ pascal seconds (Pa·s), assuming that the liquid can be considered as being water; and
the shape of the capillary (circular, triangular, or rectangular section), leading to diameters in the range 15 micrometers (μm) to 60 μm, for lengths lying in the range 1 meter (μm) to 15 μm, with a pitch of 100 μm to 150 μm between two successive turns.

In a preferred embodiment, the capillary 11 presents a section that is triangular or rectangular, the capillary space being made by putting two planes (surfaces) in contact, one of which surfaces includes a groove. This solution provides greater safety in terms of the path followed by the liquid for perfusing and its flow rate.

Under such circumstances, the coiled capillary 11 can have the following characteristics: square section with a side of about 40 μm, length lying in the range 12 m to 15 m, and a coil pitch of 120 μm.

The coil can be made of various materials, such as biocompatible plastics materials or silicon.

Prior to being put into operation, the coil 11 needs to be calibrated, which operation can be performed using a liquid (e.g. water or the medication for perfusing) or by means of a compressed gas.

The delivery rate is kept constant by the almost constant temperature of the patient's body; temperature variations lead to delivery rate variations of less than 2% per degree Celsius, and these are compatible with variations in the requirements of the user.

The device 1 of the invention is provided with control means 16 enabling the flow of liquid along the capillary 11 to be stopped and started again subsequently. Said control means comprise:
a remotely-controlled valve 13; and
valve control means (not shown) suitable for being actuated either in response to data picked up by a sensor (e.g. a glycemia sensor for diabetic patients, which sensor may be internal or external), or else as a result of manual intervention controlled from the outside. To stop or restart a system on the basis of external instructions, it is possible to use wireless transmission of commands, e.g. by radio.

This operation of stopping and restarting occurs in the event of problems (momentary hypoglycemia) or in the event of specific operations.

The electricity consumption of the valve 13 and of its control system can be provided by an electric battery of suitable dimensions or by a battery that can be recharged externally using radio waves.

A miniature solenoid valve, suitable for corresponding to the requirements of a device of the invention, is represented by way of example by the EPSV valve sold by the American supplier Lee Company, having a magnet associated therewith so as to enable it to operate in response to electrical pulses of short duration, with the valve consuming no electricity when in the open position or the closed position.

The capillary 11 also includes flow rate measuring means 14, constituted by a pre-calibrated center point pressure sensor and its conditioner. Said sensor measures the pressure of the liquid at a point situated between the inlet and the outlet of the capillary, preferably at its middle. In normal operation, the pressure is equal to a pressure lying between the pressure of the liquid in the supply and the pressure at the point of injection, which is close to atmospheric pressure. When there is no flow, the measured pressure takes on one or other of said values, depending on which half of the duct is obstructed. The pressure sensor thus serves to indicate when flow is taking place and to measure the value thereof accurately in real time.

This measurement is made on the basis of the viscosity of the liquid that is to be injected, of certain characteristics of the capillary (pressures upstream and downstream of the capillary), and of the temperature of the human body.

The pressure sensor is a device formed by a body and a membrane, with deformation thereof being measured by means of strain gauges. Such sensors adapted to use in the context of a device of the invention can be constituted, for example, by the miniature sensors sold by the American supplier Entran.

The control and monitoring system of the device of the invention thus performs two functions:
controlling the valve 13 on the basis of internally- or externally-generated orders (open or close); and
emitting a warning signal in the event of the pressure sensor detecting malfunction;
   with transmission possibly taking place under both circumstances by radio.
   Catheter

The catheter 15 is implanted in a blood vessel, in particular a central vein, preferably in a zone of high turbulence so as to avoid forming a mass of fibrin at its end. The catheter provides the connection between the supply 6 and the point of injection.

The catheter can become clogged if the length of time during which the flow along said catheter is stopped exceeds a duration of about 30 min. In order to avoid any obstruction of the catheter 15, means for monitoring the flow in the capillary 11 are integrated in the means for controlling the valve 13, enabling said valve to be restarted at regular intervals during a predefined period; specifically, it consists in installing a program in the control means for the valve 13, i.e. instructions implemented in an integrated circuit, that ensure a flow of short duration (30 seconds (s) to 1 min) restarting approximately once every 10 min, whenever the valve 3 is in the OFF position.

The catheter 15 is required to operate over long periods of time, lying in the range 5 years to 7 years; it is therefore essential for it to be properly secured to the selected central vein, so that it cannot become separated therefrom and so that no blood clot forms which could lead to head loss that might be high and lead to a decrease in the pressure difference between the ends of the circuit.

The dose of medication to be injected can be varied as a function of the weight of the patient, for example by using capillaries having higher flow coefficients for heavier patients, and by diluting the medication in order to obtain appropriate doses.

In an embodiment, the quantity of fluid to be perfused can be modified overall by varying the length of time the valve 13 is open (ON), in compliance with predefined programs, as a function of the specific needs of the patient.

The device of the invention can be used together with other existing means that are used for lowering glycemia: injections, inhalations, etc. of insulin.

What is claimed is:

1. An implantable device for administering a liquid, in particular a medication, in man or animal, the device comprising:
a transfer chamber enabling a supply to be fed with liquid;
transfer means for transferring said liquid between the transfer chamber and said supply; and
a supply enabling said liquid to be delivered, the supply including:
an inlet for receiving said liquid; and
at least one outlet for delivering said liquid to the patient.

2. A device according to claim 1, in which said outlet of the supply for delivering a liquid to the patient is connected to a capillary.

3. A device according to claim 2, in which the capillary is provided with control means enabling the flow of liquid in said capillary to be adjusted.

4. A device according to claim 3, in which said control means comprise a remotely-controlled valve and means for controlling said valve.

5. A device according to claim 4, in which said means for controlling the valve comprise an internal or external sensor.

6. A device according to claim 2, in which the capillary is provided with a catheter at its distal end.

7. A device according to claim 2, in which the capillary is provided with means for measuring the flow of the liquid in said capillary.

8. A device according to claim 7, in which said flow measurement means comprise a central point pressure sensor.

9. A device according to claim 1, in which said supply includes means for putting the liquid contained in the chamber to a constant pressure.

10. A device according to claim 9, in which said means for putting the liquid contained in the chamber to a constant pressure comprise using a phase-change fluid.

11. A device according to claim 10, in which the phase-change fluid is isobutane or propane.

12. A device according to claim 1, in which said means for transferring liquid between the transfer chamber and the supply comprise a tube and a check valve.

13. A device according to claim 1, in which said tube presents a length that enables the transfer chamber to be implanted under the skin and the supply at a distance therefrom, at a selected location in the body of the patient.

14. A device according to claim 4, in which means are provided for monitoring the flow in the capillary, integrated with the means for controlling the valve, enabling the valve to be restarted at predefined regular intervals for predefined durations.
